(19) United States
(12) Patent Application Publication
(10) Pub. No.: US 2011/0209274 A1
(43) Pub. Date: Sep. 1, 2011

(54) Form And Function Helmet

(76) Inventor: John Alexander Del Rosario, New York, NY (US)

(21) Appl. No.: 13/028,447

(22) Filed: Feb. 16, 2011

Related U.S. Application Data
(60) Provisional application No. 61/309,097, filed on Mar. 1, 2010.

(51) Int. Cl. A63B 71/10; A42B 3/22; A61F 9/00

(52) U.S. Cl. 2/425; 2/10

(57) ABSTRACT

To be manufactured, formed and operated in much different and diverse fashion as compared to conventional motorsport helmets is a strong yet flexible and lightweight safety “helmet.” Comprising a “shell,” and several manually operated “components”. To be incorporated onto said shell are numerous actuating and/or fixed mechanisms with inherent safety aspects meant to increase the comfort, peripheral vision and facilitate the overall safety of the wearer. This multiplicity of independent components is also designed to protect the user's head and cranium and to reduce the violent sudden rotational forces encountered after impact. One aspect consists of a solid, continuous and unbroken “Shell.” The shell comprises a number of curves and concave segments with several other significant distinguishable design details meant to improve the overall aerodynamics of the shell. Other components include a “main external visor” and a secondary “inner visor” that actuate in multi-step processes. Aerodynamic rear and side “spoilers/elevations”, a rear “stabilizing/grab bar” for rotational force attenuation and two air “centrally mounted intake ducts”/“exhaust vents” for wearer ventilation are components that would normally be attached to this shell via commercially available adhesives and or fittings. A flush convergence of parts and components designed to work in balanced form. Presented is a much more low-profile product with a much smaller cross section in comparison to the given norm. The end result is a revolutionary product in both function and form.

### BACKGROUND OF THE INVENTION

This invention relates generally to protective headgear and, more specifically, to a distinguishing shaped and operated motorsports helmet that improves visibility, comfort and safety to the user.

Protective headgear or helmets have been worn for a long time now by individuals to protect against head injuries. The use of helmets is often a mandatory requirement for driving bicycles and certain other motor vehicles in high impact sports and in material handling and other potentially hazardous locations.

The use of safety helmets has been just that—to reduce or completely protect the user from any top, lateral and penetration impact to the user's head. However, very little to no changes have come about in form or functionality or external design within the past 50+ years. Commonly used protective headgear use a hard outer casing with an impact-energy absorbing padding placed between the outer casing and the user's head. These hard casing helmets would normally consist of the hard outer shell, a visor and air intake/exhaust ports. Designed in such a way to facilitate mass manufacturing methods and cost-cutting measures. Traditional helmets usually have a much more pronounced jaw section with a decreasing diameter as you approach the top, this is not by any means a cosmetic choice by the manufacturers, this is a result of function due to regulatory restrictions. What you get is a terribly unbalanced and “Unnatural” anthropometric shape. Should they attempt to bring the frontal area forward, it would severely affect it's already Uneven center of gravity, thus making the already “Top-heavy” helmet unbearable to withstand and unfit to wear. In terms of graphics and colors, the way manufacturers normally make the helmets is by applying graphic work to the shell and leaving the face shield either clear or tinted with a solid color.

Although these and other conventional helmet designs have worked well, they have failed to take into account the general dynamics in human form and/or human behavior in general. Also overlooked are new and improved design and manufacturing methods. While the motorsport and safety industry in general has evolved and improved, the helmet industry although profitable has become stagnant and mundane during all these years.

The flaw in a number of many designs is the tendency to become too similar and indistinguishable. Throughout the years there have been numerous design, color and cosmetic add-ons applied to this basic shape. Despite these efforts they've remained just that, ornamental. Very little to no significant changes have come about in terms of the basic shape and function.

U.S. Pat. Nos. D445546 to Gafforio, D331299 to Kamata and D461935 to Hamn respectively disclose just a few examples in a variety of takes on the same usual form in safety helmet design for motorcyclists. An outer shell of the helmet is formed with much in common to many other ornamental helmet designs with just slight enough variation to warrant a patent issue. Crash helmets disclosed by these patents do not seem likely to provide a significant improvement over current safety helmet shells.

U.S. Pat. Nos. D498563 to Fournier & Bourgeois, 6,006,366 to Vondrak and D497223 to Kalhok all disclose helmet visor mechanisms that function with much the same movements but differentiate themselves in the execution of the design.

U.S. Pat. No. D485,948 to Yi discloses a protective helmet with a sculpted rear most likely for aerodynamic function. This feature is seemingly inherent to the shell of the helmet. Also disclosed within this design are the dual air intakes/exhaust ports that are both considerably spaced apart and both begin and end on top of the shell. Meaning the intake/exhaust ports and the rear spoiler section are not joined together.

U.S. Pat. No. D621551 to Isobe illustrates a safety helmet with a prominent rear spoiler protruding beyond the shell. Note that this spoiler also does not attach to the intake/exhaust vents and are both separate and independently attached to the main shell. Also note that this configuration may also be a hindrance and a possible injury risk with consideration to rotational forces encountered during a rear head impact. This protrusion would cause an uneven balance with the top and the bottom of the helmet causing the wearer's head to move in unintended directions. Thus, this configuration would provide no possible benefit to the wearer.

U.S. Pat. No. 489491 to Corser illustrates one of the many typical design/paint schemes applied to motorcycle helmets. Normally a paint or design pattern is layered onto the shell and it's components with the exception to the visors. Conventional helmet visors would normally be tinted in one solid color or have no tint whatsoever. The case being, conventional helmets and helmet visors do not share the same paint patterns.

All these aforementioned helmets do not seem likely to provide a significant improvement over current safety helmet shell design and function.

Hence, it is the object of the present invention to overcome the aforementioned problems and create a novel and improved, versatile and innovative motorcycle helmet shell.

### BRIEF SUMMARY OF THE INVENTION

Accordingly, the present invention strives to overcome some of the disadvantages of prior safety helmet designs by a) providing a component system that minimizes weight and size as to be more faithful in proportion to the user's anatomical head, thereby minimizing resultant disproportion between the head with helmet and the neck/torso and by b) redirecting or dissipating injurious rotational forces away from the head and brain, by using an external stabilizing structure. C) Revolutionizing the helmet shape to better lower the helmets center of gravity and improving its aerodynamic qualities. D) Designing an air induction/exhaust system that improves overall ventilation and hot air release within the helmet for wearer comfort. Lastly e) incorporating flush graphics through special color tinting processes that would allow for the wearer to have visual clarity and improved peripheral vision.

An outer shell structure herein referred to as “shell” and multiple components affixed onto the shell via commercially available adhesives and or coupling methods herein referred to as “components,” collectively referred to as “form and function helmet” or “helmet.” This helmet is particularly for facilitating wearer comfort and ease of use. The helmet comprises a number separate components adhered to the shell in final assembly. The main component being the shell, additional components being double air intake ports” and double “exhaust vents” that run the length of the helmet back to front. These air intake ports and exhaust vents would normally be attached to the rear spoiler of the helmet. This spoiler component helps in the cooling and ventilation of the wearer while facilitating airflow throughout the top of the shell during high-speed motorsport activities.

Another component is the “main external visor.” Movement of this visor is actuated via the wearer manually pushing the front of the visor inwards thus causing the visor to spring forward and upwards.

A secondary “inner-visor” is to be embedded within the liner and shell of the helmet. This inner visor also has an unconventional movement as compared to other helmets in the motorsports segment. The inner-visor is actuated via the wearer manually moving the visor in a horizontal “side-to-side” motion as opposed to the vertical “up and down” movement in conventional motorcycle helmets.

A few of the other important practical components included into the helmet design are numerous aerodynamic spoilers and elevations shaped to correspond to the airflow within the areas they are covering respectively: These minor cues are quite possibly one of the most important aspects in the design of each helmet. Each helmet will inevitably contain many openings and rough edges in order to provide for ventilation or movement of mechanical parts. This presents a slight dilemma. Causing drag and wind noise, these openings and hard edges need to have a sort of “buffer” to redirect the wind and prevent the wearer from encountering severe wind drag and extremely high-pitched levels of wind noise at high speeds. Thus, these “elevations” provide a sort of “airfoil” effect on the helmet, redirecting the airflow over and around the respective area.

Numerous indents and concave features are designed into the shell itself. Indeed an aesthetic and aerodynamic design cue but no less important in terms of functionality, these indents are there to facilitate the opening and closing of certain sections of the helmet without resorting to straight lines or rough edges seen in most shell designs.

The lower rear arch is used as a “stabilizing/grab bar” of sorts to aid in wearer safety when encountering rotational forces during an impact. Because of the noticeably extended protrusion of the rear spoiler, a counterbalance is needed so as to not injure the wearer during an impact event. This “stabilizing/grab bar” is also beneficial for the ease of helmet transport. It may be used as a grip or handling component. It is aesthetic in many ways and can also function as a way to secure and carry the helmet in certain situations.

A significantly noticeable detail when looking at the design are the prominent side encasements. Conventional helmets usually integrate a thermoplastic cover to conceal the visor mechanisms. This new design however, incorporates a more complex structure given the complexity of both the inner-visor and external visor. The “temporal side encasements” are an aesthetic and functional piece of kit meant to improve and facilitate the ease of use when handling these components.

Lastly, the flush “graphics” as a feature is a modern yet revolutionary color tinting and paint process whereby the graphics run continuous throughout the entirety of the helmet. Usually, the way manufacturers make the helmets is by applying graphic work to the shell and leaving the face shield either clear or tinted with a solid color. Through a process in which a high-temperature Fine Line Masking Tape is applied. A very thin film backed tape for the most critical paint break lines is used to apply tint on the desired areas of the shield and thus merge the graphics from the shell and visor sections together to create a seamless design. Masking of curved or irregularly shaped surfaces may be accomplished by using die-cut parts made from these specialty tapes. This process would still allow a wearer to be able to see clearly and through specified degrees of curvature and not the least of which being able to clearly see the colors in a stoplight without difficulty.

A practical consideration of all these components in the helmet is that it allows the wearer to act instinctively and with minimal effort as possible use the helmet while maintaining their full attention to the road.

These and other objects of the invention will be apparent from the following drawings and detailed description of the invention.

### BRIEF DESCRIPTION OF THE SEVERAL VIEWS OF THE DRAWINGS

FIGS. 1A & 1B are side elevational views illustrating both “filled-in” and outline thereof,

FIGS. 2A & 2B are three-quarter side elevational views thereof,

FIGS. 3A & 3B are front three-quarter elevational views illustrating both “filled-in” and outline thereof;

FIGS. 4A & 4B are rear elevational views illustrating both “filled-in” and outline thereof;

FIGS. 5A & 5B are three quarter side elevational views thereof,

FIG. 6 is a top plan view thereof.

FIG. 7 is a side elevational view thereof

FIG. 8 is a perspective frontal view thereof.

### DETAILED DESCRIPTION OF THE INVENTION

Referring now to the drawings wherein like or corresponding reference numerals are used for like or corresponding parts throughout the several views. The present invention incorporates a shell 1, herein referred to as “shell”, This shell firmly coupled with numerous components on its surface herein referred to as “components” No's 2, 3, 4, 5, 6, 7, 8, 9, 10, 11, and 12, herein referred to as “components.” Said assembly herein collectively referred to as “helmet.” The helmet may be manufactured in separate parts and processes then integrated during final assembly.

In a preferred embodiment a strong yet flexible helmet shell 1 would be the first main part. This part is thus contoured and designed to comply with certain comfort and aerodynamic parameters for optimal wearer comfort.

In a preferred embodiment the outermost layer or “shell” 1 can be made up of a strong yet flexible construction preferably of fiber-reinforced composites or thermoplastics or the like with the unique quality of minimal sliding friction. The reason for this material having a low friction coefficient is that the helmet is supposed to slide along, i.e. move the head of the user along with the rest of the body. Researchers have remarked that while the helmet should protect the user's head from impact forces, the helmet (when in contact with the ground or any other large object) should not impede or resist the movement of the head as compared to the rest of the body, which might be carrying or moving forward with a good momentum when the user has fallen off a moving vehicle or other impact event. Such a restriction to the movement of the user's head vis-a-vis the user's body had shown detrimental results with damages to the neck and head region of the user—as the body would be moving with a higher momentum and if the head's momentum is slowed by the helmet it would induce severe stress on the neck region. As such, the outer layer 1 would be made of a material that would protect the user's head from the impact forces yet have a very low friction coefficient with potential contact surfaces.

In a preferred embodiment the shell 1 would be designed as to have a forward-angled frontal section 2, the top frontal area of the helmet has been angled forward in comparison to conventional helmet design while the lower jaw section has been pulled back in in order to maintain continuity in the resulting arc. It is shaped to follow the more “Natural” and symmetrical contours of the human skull. This allows for a more evenly distributed weight ratio and a lowered center of balance. The result is a much more ergonomically pleasing and anthropomorphic form to the helmet. This revolutionary shape also allows for an aerodynamically superior shape, greatly reducing lift and drag and dramatically increasing airflow around the ventilation intakes. The design is geared towards a cleaner and uncluttered look.

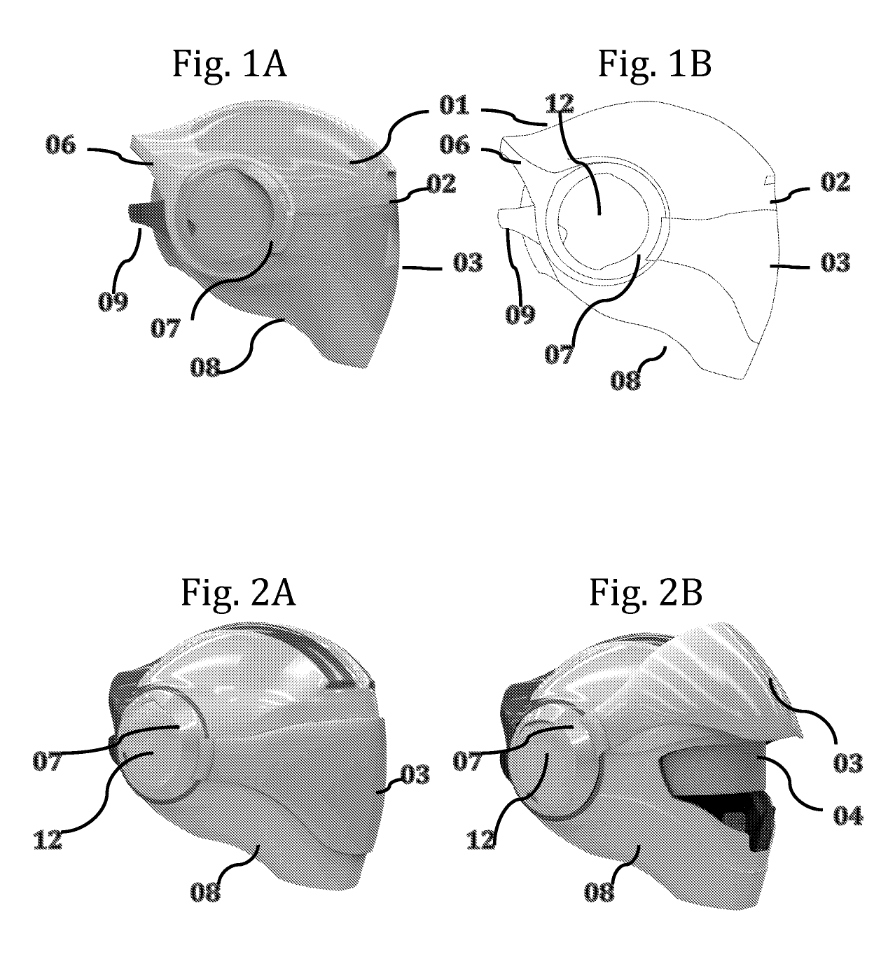

In a preferred embodiment the main external visor 3 is also shaped in a unique fashion as to follow and fit closely with the unique lines of the helmet FIGS. 1A & 1B, 2A & 2B, 5A & 5B, 7 and 8. This part 3 is thus contoured and designed to comply with certain comfort and aerodynamic parameters 2.

In a preferred embodiment a secondary “inner-visor” 4 is to be embedded within the liner and shell of the helmet FIGS. 2A & 2B, 3A & 3B. This inner visor also has an unconventional movement as compared to other helmets in the motorsports segment. The inner-visor is actuated via the wearer manually moving the visor in a horizontal “side-to-side” motion as opposed to the vertical “up and down” movement in conventional motorcycle helmets.

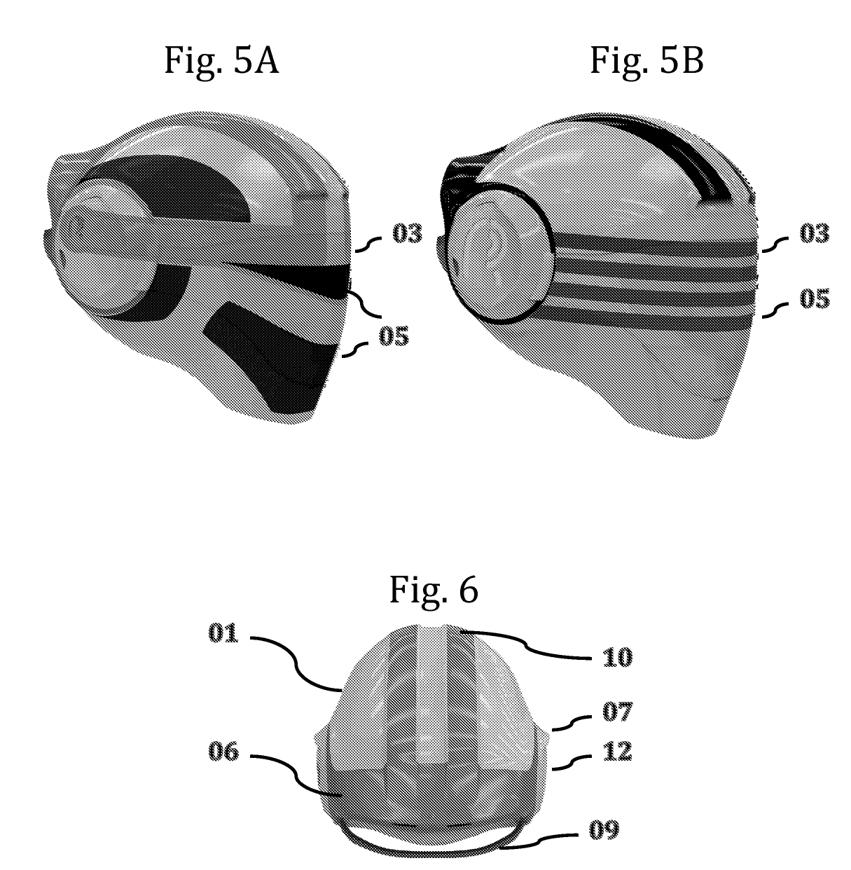

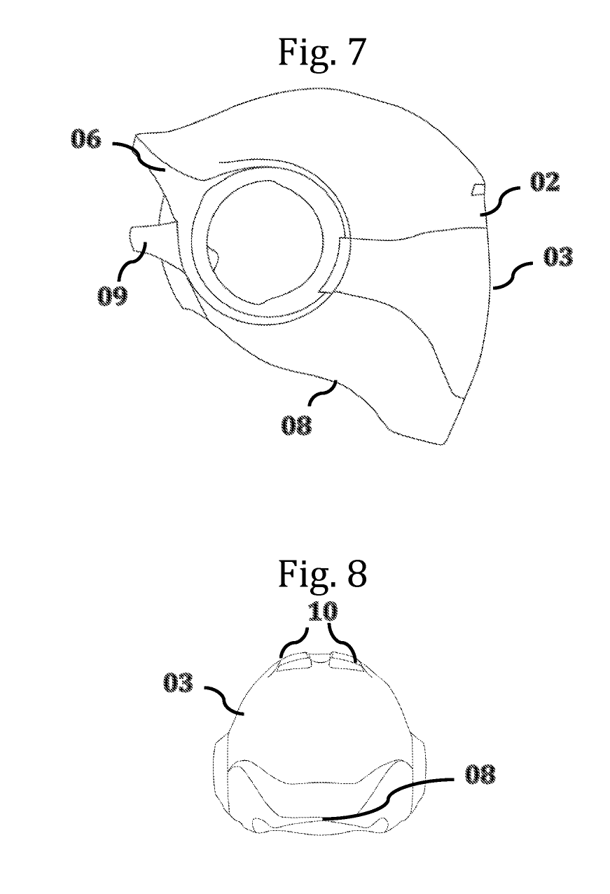

There is shown in FIGS. 5A & 5B the preferred embodiment of separate views of one significant part of the present invention. Consisting of continuous and flush graphics 5 that run the length of the helmet back to front. This feature has a simplified yet revolutionary color scheme where the graphics run continuous throughout the entirety of the helmet. Through a process in which a high- temperature Fine Line Masking Tape is applied. A very thin film backed tape for the most critical paint break-lines is used to apply tint on the desired areas of the shield and thus merge the graphics from the shell and visor sections together to create a seamless design. Masking of curved or irregularly shaped surfaces may be accomplished by using die-cut parts made from these specialty tapes. This process would still allow a wearer to be able to see clearly and through specified degrees of curvature and not the least of which being able to clearly see the colors in a stoplight without difficulty. The graphics 5 may comprise a plurality of designs that extend over and about the helmet.

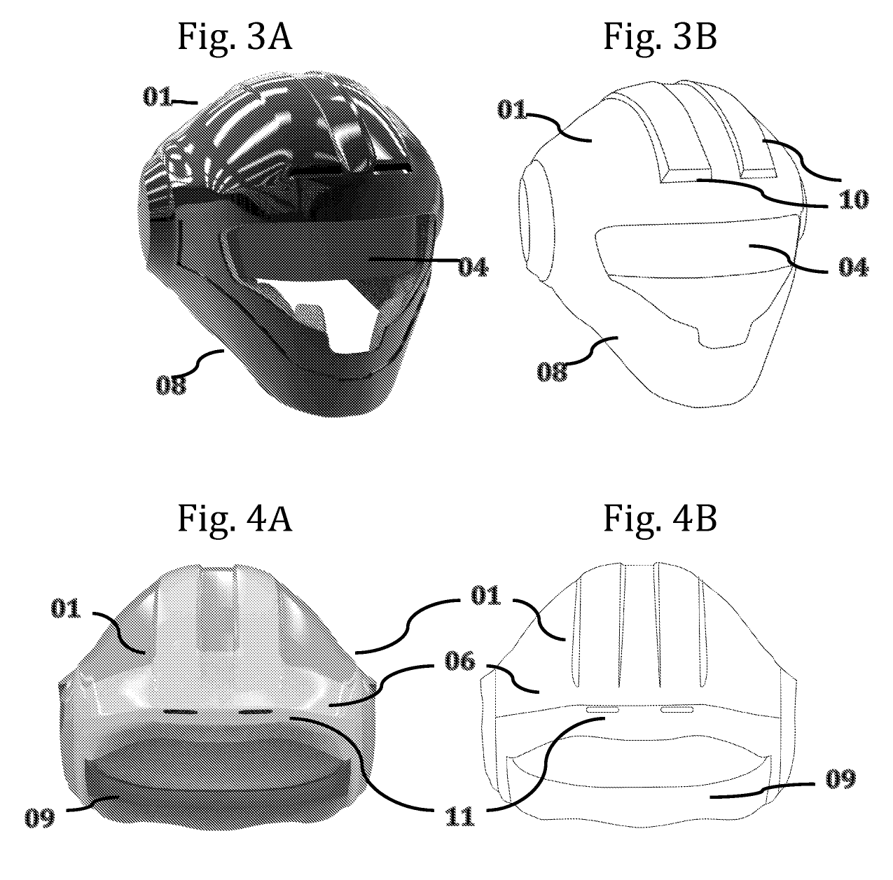

In a preferred embodiment as shown in FIGS. 3A & 3B, 6 & 8, the air intake ports 10 provide a way for the wearer to receive ventilation while wearing the helmet. Due to the helmet shell's unique design, these air intake ports are centrally located on the top of the helmet in a closer configuration and in a more symmetrical fashion. This central location atop the head FIG. 8 and closer together would be better situated to receive utmost airflow while retaining great aesthetic value. They are placed in such a way that they are symmetrically proportional to the eye-lines of the human face FIG. 8. The sections are spaced apart to just enough provide for even ventilation throughout the human head, with spacing between them of approximately but not limited to one inch or more. Thus, adequate ventilation is provided to dissipate heat and achieve a reasonable level of comfort while protecting the wearer. In another preferred embodiment, the width of a section is to be approximately but not limited to three quarters of one inch.

In a preferred embodiment the exhaust vents 11 improves overall ventilation and hot air release within the helmet for wearer comfort FIGS. 4A & 4B. These vents would preferably be located within the rear spoiler 6.

In a preferred embodiment he rear spoiler 6 provides drag reduction at high speeds. This design however incorporates the rear spoiler into the overall aesthetic design. The large rear spoiler along with the other spoiler/elevation attachments 7 & 12 are quite possibly one of the most important aspects in the design of each shell. Each helmet will inevitably contain many openings and rough edges in order to provide for ventilation, which presents a slight dilemma. Causing drag and wind noise, these openings and hard edges need to have a sort of “buffer” to redirect the wind and prevent the wearer from encountering severe wind drag and extremely high-pitched levels of wind noise at high speeds. Thus, these “elevations” provide a sort of “airfoil” effect on the helmet.

While in a preferred embodiment the large rear spoiler 6 and the air intake ports 10 can be combined into one solid component, he remaining spoiler components 7 & 12 are each independent of each other and not attached or manufactured jointly with any other part.

In a preferred embodiment the “temporal side encasements” 12 are an aesthetic and functional piece of kit meant to improve and facilitate the ease of use when handling the multiple visor attachments and fittings FIGS. 1A & 1B, 2A & 2b and 6. Providing a secure mechanism cover while simultaneously covering certain elements from wind turbulence.

In a preferred embodiment the concave sections”/”indents” 8 are there to facilitate the opening and closing of certain sections of the helmet without resorting to straight lines or rough edges seen in most shell designs FIGS. 1A & 1B, 2A & 2B, 3A & 3B, 7 and 8. These sections are also shaped and contoured to follow and conform to certain wearer comfort concerns and aerodynamic parameters for optimal wearer comfort.

While in a preferred embodiment the aforementioned helmet may be formed with fiber-reinforced composites or thermoplastics or the like. The shell and its components may be used in conjunction with other commercially available polymers and plastics to form the segments of the present invention. In addition to polystyrene or polypropylene, the segments may be made from, without limitation, a polybutylene, a polyvinyl (including polyvinyl chloride), a polyester, a polycarbonate, a polyurethane, a polyamine, a polyacrylic, a polyamide, a polyurea, and any other suitable polymer.

In the foregoing specification, the invention has been described with reference to an illustrative embodiment thereof. However, it will be evident that various modifications and changes may be made thereto without departing from the broader spirit and scope of the invention. The specification and drawings are, accordingly, to be regarded in an illustrative rather than a restrictive sense. Therefore, it is the object of the appended claims to cover all such modifications and changes as come within the true spirit and scope of the invention.

Although the preferred embodiment may at certain points describe the construction as preferably a motorsports safety item, the invention is not so limited. It is to be appreciated that the system construction of the present invention could be modified for almost any sports or non-sports application where a protective head covering could be required, including without restriction its use as a horseback riding helmet, construction helmet, football helmet, skateboard or snowboard helmet, a motorcycle or race car driver helmet, and the like.

1. I hereby claim a safety helmet to be constructed and assembled with distinguishable form and mechanical function herein referred to as “form and function helmet” or “helmet”. Comprising a plurality of independent components and attachments herein referred to as “components.” All combined and designed to protect the user's head and cranium and to reduce the violent sudden rotational forces of the head and brain after impact. This helmet is also designed to aid in better peripheral vision and ease of use to the wearer via manually actuated automated movements. A strong yet flexible and lightweight helmet “shell” herein referred to as “shell.” Comprising of a “main external visor” herein referred to as “Main external visor” and a secondary “inner-visor” herein referred to as “inner-visor”. Also comprising of an innovative process whereby “paint and or graphics” herein referred to as “flush graphics” are continuous and unbroken throughout the entirety of the helmet. Also, comprising of a prominent rear “aerodynamic spoiler” herein referred to as “rear spoiler”. Also comprising of “aerodynamic side spoilers and elevations” herein referred to “side spoilers/elevations.” Also comprising of specially designed “concave sections or indents” herein referred to as “ concave sections/indents.” Also comprising of a lower arched panel or “stabilizing bar” herein referred to as “stabilizing bar” utilized for attenuating rotational forces during an impact and to facilitate handling of the helmet. Also comprising air intake ports and exhaust vents herein referred to “air intake ports” and “exhaust vents” designed to increase overall ventilation and comfort to the wearer. Lastly, comprising of temporal side encasements herein referred to “Temporal side encasements” used to safety contain the mechanisms used to actuate visor movement. The helmet may be manufactured as an integrated, standalone protective layer that could be universally adapted and incorporated onto any modifiable safety design shaped to correspond with required design parameters and the user's head.

2. The invention according to claim 0001 wherein one component consists of a solid, continuous and unbroken “shell.” Said strong yet flexible helmet shell is thereby designed and contoured to fit the parameters of the human form and desired functions.

3. The invention according to claim 0002 wherein the outermost layer or “shell” can be made up of a strong yet flexible construction preferably of fiber-reinforced composites or thermoplastics or the like with the unique quality of minimal sliding friction.

4. The invention according to claim 0003 wherein the shell comprises a plurality of semi-circular arched segments or concave sections and indents which are shaped to facilitate aerodynamics, weight reduction and balance.

5. The invention according to claim 0002 wherein there is a forward angled front section that extends about the Frontal region of the skull.

6. The invention according to claim 0002 wherein there is a reduced protrusion of the jaw section on the lower frontal base region of the shell.

7. The invention according to claim 0006 wherein said forward angled front section is a forward angled arc continuous and unbroken extending onto the frontal base of the helmet shell.

8. The invention according to claim 0001 wherein there is a forward then vertically actuating main external visor.

9. The invention according to claim 0008 wherein this main external visor is operated whereby the operator manually pushes the visor inwards, hence actuating a visor movement in which the main external visor pushes outward and upward onto it's final open position

10. The invention according to claim 0001 wherein there is a secondary inner visor.

11. The invention according to claim 0010 wherein said inner visor extends horizontally about the frontal region of the wearer's head.

12. The invention according to claim 00011 wherein said inner visor movement is actuated manually by the wearer whereby the inner visor moves in a horizontal direction as opposed to vertically.

13. The invention according to claim 0001 wherein there are flush graphics applied onto the helmet. Said graphics on the helmet are painted and or tinted via a special process whereby the graphics run continuously throughout the entirety of the helmet without having separation between main external visor and shell.

14. The invention according to claim 0013 wherein this process includes but is not limited to where a high-temperature Fine Line Masking Tape is applied to cover the non-tinted areas. Tint or paint is then applied. Masking of curved or irregularly shaped surfaces may be accomplished by using die-cut parts made from these specialty tapes.

15. The invention according to claim 0014 wherein said graphics may comprise a plurality of designs that extends over and about the helmet.

16. The invention according to claim 0001 wherein there is a prominent aerodynamic rear spoiler.

17. The invention according to claim 0016 wherein there are two centrally mounted air exhaust vents used to facilitate the release of ventilation throughout the helmet.

18. The invention according to claim 0001 wherein there are aerodynamic side spoilers and elevations.

19. The invention according to claim 0001 wherein there is a horizontally oriented arch or stabilizing grab/bar used to stabilize rotational forces during an impact event.

20. The invention according to claim 0019 wherein said stabilizing/grab bar may be used in the handling of the helmet during transport.

21. The invention according to claim 0001 wherein there are two centrally mounted air intake ports used to facilitate ventilation to the wearer.

22. The invention according to claim 0021 wherein said air intake ports are spaced apart to provide for ventilation, with spacing between them of approximately but not limited to one inch or more. Thus, adequate ventilation is provided to dissipate heat and achieve a reasonable level of comfort while protecting the wearer.

23. The invention according to claim 0020 wherein the width of a panel is to be approximately but not limited to three quarters of one inch.

24. The invention according to claim 0001 wherein there are temporal side encasements meant to encase mechanisms used in visor actuation.

25. The invention according to claim 0001 wherein in a preferred embodiment, said components are bonded or held together by way of commercially available processes.

26. The invention according to claim 0025 wherein said processes may include but are not limited to applying commercially available adhesive compounds.

27. The invention according to claim 0031 wherein in a preferred embodiment of the invention, the layers are made of polystyrene or polypropylene, the aforementioned may be used in conjunction with other polymers and plastics to form the segments of the present invention. In addition to polystyrene or polypropylene, the segments may be made from, without limitation, a polybutylene, a polyvinyl (including polyvinyl chloride), a polyester, a polycarbonate, a polyurethane, a polyamine, a polyacrylic, a polyamide, a polyurea, and any other suitable polymer.

28. The invention according to claim 0001 wherein the aforementioned helmet may be formed with fiber-reinforced composites or thermoplastics or the like. The shell and its components may be used in conjunction with other commercially available polymers and plastics to form the segments of the present invention. In addition to polystyrene or polypropylene, the segments may be made from, without limitation, a polybutylene, a polyvinyl (including polyvinyl chloride), a polyester, a polycarbonate, a polyurethane, a polyamine, a polyacrylic, a polyamide, a polyurea, and any other suitable polymer.

29. The invention according to claim 0001 wherein the helmet has been described with reference to an illustrative embodiment thereof. However, it will be evident that various modifications and changes may be made thereto without departing from the broader spirit and scope of the invention. The specification and drawings are, accordingly, to be regarded in an illustrative rather than a restrictive sense. Therefore, it is the object of the appended claims to cover all such modifications and changes as come within the true spirit and scope of the invention.

30. The invention according to claim 0001 wherein at certain points the helmet construction may be described as intended preferably as a motorsports safety item, the invention is not so limited. It is to be appreciated that the construction of the present invention could be modified for almost any sports or non-sports application where a protective head covering could be required, including without restriction its use as a horseback riding helmet, construction helmet, football helmet, skateboard or snowboard helmet, a motorcycle or race car driver helmet, and the like.
